(19) United States
(12) Patent Application Publication
(10) Pub. No.: US 2006/0024847 A1
(43) Pub. Date: Feb. 2, 2006

(54) Controlled optoelectronic coupling in nanoparticle arrays

(75) Inventors: Fazila Seker, Clifton Park, NY (US); Patrick Roland Lucien Malenfant, Clifton Park, NY (US); Azar Alizadeh, Wilton, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/157,548

(22) Filed: Jun. 21, 2005

Related U.S. Application Data
(60) Provisional application No. 60/592,629, filed on Jul. 30, 2004.

(51) Int. Cl. G01N 33/545

(52) U.S. Cl. 436/531

(57) ABSTRACT

In some embodiments, the present invention is directed to methods by which nanoparticle interactions can be controlled, compositions with which such interactions can be controlled, and devices which utilize the control of such interactions. Generally, such methods involve grafting polymer to electromagnetically-functional cores to form a core/shell nanoparticle, assembling a plurality of such core/shell nanoparticles to form an assembly, and exposing the assembly to at least one environmental stimulus to which the polymer is responsive so as to modulate the interparticle interactions of the electromagnetically-functional cores. The present invention is also directed to the compositions resulting from such methods and to the methods and associated devices for controlling the interparticle interactions in such compositions.

### CROSS-REFERENCE TO RELATED APPLICATIONS

This Application claims priority to U.S. Provisional Patent Application Ser. No. 60/592,629, filed Jul. 30, 2004.

### TECHNICAL FIELD

The present invention relates generally to nanoparticulate interactions, and more specifically to methods by which nanoparticle interactions can be controlled, compositions in which such interactions can be controlled, and devices which utilize the control of such interactions.

### BACKGROUND INFORMATION

Quantum dots have captured the imagination of generations of scientists because they afford the ability to continuously vary optoelectronic properties with particle size, thereby opening up new material design spaces. The electromagnetic phenomena that characterize these systems are highly sensitive to a hierarchy of spatial dimensions, not just particle size (Farbman et al., J. Phys. Chem., 1992, 96:8469; Farbman et al., J. Chem. Phys., 1992, 96:6477). While many synthetic techniques have been developed for creating a narrow particle size distribution at a targeted particle size, few techniques exist for predictably and precisely controlling the relative positioning of particles with respect to one another, a critical requirement for controlling interparticle coupling, and therefore system properties (Weller, Angew. Chem. Int. Ed. Engl., 1996, 35:1079; Murray et al., Science, 1995, 270:1335; al., J. Am. Chem. Soc., 2000, 122:4640; and Redl, Nature, 2003, 423:968). Consequently, control of interparticle interactions has been the key bottleneck to implementing quantum dots in a number of applications.

U.S. Pat. No. 6,544,800 (Asher) describes a sensor device that is a crystalline colloidal array of charged particles polymerized within a hydrogel matrix. The crystalline colloidal array is typically colloidal polystyrene self-assembled in the matrix. The self-assembled colloidal array diffracts light in response to stimulus-induced volume changes in the hydrogel matrix. The sensors of Asher use large (>100 nm) particles that are not electromagnetically functional. In addition, Asher's method of preparation is not applicable to small (<100 nm) functional particles.

Gold nanoparticles have been coated with a thin monolayer of a specific hydrogel polymer by Shan et al. in Macromolecules, 2003, 36:4526-4533. Such prior work has included the synthesis of gold colloids in the presence of poly(N-isopropylacrylamide) pNIPA and surface-initiated growth of pNIPA has also been reported. These particles, however, were found to be only slightly soluble in water, leading to uncontrolled aggregation—even in very dilute solution (Shan et al., Macromolecules, 2003, 36:4526; Raula et al., Langmuir, 2003, 19:3499). Furthermore, no assembly into films has been reported.

As a result of the foregoing, a method and/or composition by/with which nanoparticle interactions can be conveniently and effectively controlled would be extremely useful, particularly when such methods and/or compositions overcome the limitations of the prior art, and where the methods and/or compositions can be used in the fabrication of sensors and other similar devices.

### BRIEF DESCRIPTION OF THE INVENTION

In some embodiments, the present invention is directed to methods by which nanoparticle interactions can be controlled, compositions with which such interactions can be controlled, and devices which utilize the control of such interactions. Generally, such methods involve binding and/or grafting polymer to electromagnetically-functional cores to form a core/shell nanoparticle, assembling a plurality of such core/shell nanoparticles to form an assembly, and exposing the assembly to at least one environmental stimulus (e.g., heat, light, moisture, etc.) to which the polymer is responsive so as to modulate the interparticle interactions (i.e., electromagnetic coupling) of the electromagnetically-functional cores. The present invention is also directed to the compositions resulting from such methods and to the methods and associated devices for controlling the interparticle interactions in such compositions.

In some embodiments, the present invention is directed to a method comprising the steps of: (a) providing a plurality of electromagnetically-functional cores; (b) providing a polymeric shell to each of the electromagnetically-functional cores to form a plurality of core/shell composite nanoparticles, wherein the polymeric shell is responsive to at least one environmental stimulus, and wherein the polymeric shell is bound to the electromagnetically functional core in a manner selected from the group consisting of non-specific binding at sites along the length of the polymeric chain, end-grafting involving moieties at the ends of the polymer chains, and combinations thereof; (c) assembling the plurality of composite nanoparticles into an assembly in which the electromagnetically-functional cores are subject to being electromagnetically coupled to each other; and (d) exposing the continuous assembly to at least one environmental stimulus so as to modulate the extent to which the electromagnetically-functional cores are electromagnetically coupled to each other.

In some embodiments, the present invention is directed to a core/shell composite nanoparticle with controlled polymer shell architecture or binding. The composite nanoparticle comprises: an electromagnetically-functional core having a diameter in a range from about 1 nm to about 100 nm; and a polymeric shell disposed on an outer surface of the electromagnetically-functional core and substantially covering the electromagnetically-functional core. The polymer comprising the shell is typically derivatized with an appropriate chemical functionality for directing the desired architecture or binding mode. The controlled polymeric shell architecture, may, for example, be one in which the polymer shell is end-grafted to the core, or non-specifically bound to the core through multiple points along the length of the polymer chain, or bound to the core by both end-grafting and non-specific binding. The polymeric shell is typically responsive to at least one environmental stimulus.

In some embodiments, the present invention is directed to a film, wherein the film comprises: a stabilized polymeric matrix, and wherein the stabilized polymeric matrix is responsive to at least one environmental stimulus; and an assembly of electromagnetically-functional cores disposed in the matrix. Each of the electro-magnetically functional cores has a diameter in a range from about 1 nm to about 100 nm. The electromagnetically-functional cores are typically monodisperse, substantially unagglomerated, and electromagnetically coupled to each other. The stabilized polymeric matrix controls an interparticle separation between the electromagnetically-functional cores throughout the film, wherein, in some embodiments, the minimum separation between core surfaces achievable by the polymer shell upon stimulation is of up to about 50 nm. In some embodiments, the minimum separation is up to about 20 nm, in some embodiments it is up to about 10 nm, and in some embodiments it is up to about 5 nm.

In some embodiments, the present invention is directed to a method of making a film. The film comprises: a stabilized polymeric matrix, wherein the stabilized polymeric matrix is responsive to at least one environmental stimulus; and an assembly of electromagnetically-functional cores disposed in the matrix. Each of the electromagnetically-functional cores has a diameter in a range from about 1 nm to about 100 nm. The electromagnetically-functional cores are typically monodisperse (but not necessarily so), substantially unagglomerated, and subject to being electromagnetically coupled to each other, and the stabilized polymeric matrix controls an interparticle separation between the electromagnetically-functional cores throughout the film. The method comprises the steps of: providing a plurality of electromagnetically-functional cores; providing a polymeric shell to each of the electromagnetically-functional cores to form a plurality of nanoparticles; assembling the plurality of nanoparticles into an assembly in which the electromagnetically-functional cores are subject to being electromagnetically coupled to each other; and stabilizing the assembly to form the film.

In some embodiments, the present invention is directed to a sensor comprising a film. The film comprises a stabilized polymeric matrix that is responsive to at least one environmental stimulus and an assembly of electromagnetically-functional cores disposed in the matrix. Each of the electromagnetically-functional cores has a diameter in a range from about 1 nm to about 100 nm. The electromagnetically-functional cores are typically substantially unagglomerated and subject to electromagnetic coupling with each other. The stabilized polymeric matrix controls interparticle separation (and associated electromagnetic coupling) between the electromagnetically-functional cores throughout the film. The sensor inspects a wavelength of radiation that is either absorbed, or emitted by the particle or sensor through luminescence, or reflected through metallic-like reflectivity, by the film. The wavelength is inspected both before and after exposure to the stimulus, wherein a change in the wavelength and/or intensity of the radiation absorbed, luminesced, or reflected by the film indicates the presence of the stimulus. In various embodiments, illustrative external stimuli comprise at least one of moisture, temperature, voltage, pH, electrical stimuli, ionic strength and other chemical stimuli, light intensity, and the like.

The foregoing has outlined rather broadly the features of the present invention in order that the detailed description of the invention that follows may be better understood. Additional features and advantages of the invention will be described hereinafter which form the subject of the claims of the invention.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 depicts, in stepwise fashion, a method for forming assemblies of electromagnetically-functional cores in a polymer matrix, where the polymer matrix is responsive to at least one environmental stimuli, in accordance with some embodiments of the present invention;

FIG. 2 depicts an assembly comprising electromagnetically-functional cores in a polymer matrix, where the polymer matrix is responsive to at least one environmental stimuli, in accordance with some embodiments of the present invention;

FIGS. 3A and 3B depict a sensor utilizing an assembly (film) of electromagnetically-functional cores in a polymer matrix, where (A) depicts an interaction of such an assembly with EM radiation in the absence of a stimuli to which the polymer matrix is responsive, and where (B) depicts the interaction of such an assembly with EM radiation in the presence of such a stimuli such that the interacted radiation of (A) differs in wavelength/frequency from that of (B), in accordance with some embodiments of the present invention;

FIG. 4 depicts thermally induced changes in interparticle spacing for a film comprised of gold/pNIPA core/shell composite nanoparticles, in accordance with some embodiments of the present invention;

FIGS. 5A and 5B depict TEM images of the gold/pNIPA core/shell particles showing that they are well dispersed (A), with the core/shell structure more clearly seen in the images to the right (B);

FIG. 6 depicts two architectures by which an electromagnetically-functional core can be encased by polymer, wherein a non-specific binding architecture is depicted by I, and wherein a graft-to architecture is depicted by II;

FIG. 7 illustrates two different sulfur-based chain-end functionalities, thiol for pNIPA-SH, and disulfide for pNIPA-SS, useful in driving the end-grafted architecture, II, in accordance with some embodiments of the present invention;

FIG. 8 depicts the polymerization of N-isopropylacrylamide using (4) as the initiating chain transfer agent to yield pNIPA-SS, in accordance with some embodiments of the present invention;

FIG. 9 depicts the effect of temperature on the average hydrodynamic diameter of core/shell gold/pNIPA particles as measured by DLS for three different surface functionalizations of gold particles (as indicated to the right), where for each derivative, the thermally-induced size change upon cycling between 22° C. and 40° C. in aqueous solution is shown, where the double-headed arrows indicate reversibility, and where the measured coil size of free 18K pNIPA-SH at 22° C. is shown for comparison;

FIGS. 10A and 10B illustrate the effect of molecular weight on the stability of pNIPA-SH adsorption on gold nanoparticles for (A) MW=10K, and (B) MW=18K;

FIGS. 11A-11C illustrate the stimulus-induced optical response of a stabilized film of gold/15K pNIPA-SS core/shell nanoparticles on a quartz slide where a color change occurs upon heating or drying the film (A), and where the encircled region in the right panel demonstrates the reflective appearance of the film when in the purple state, as well as corresponding UV-vis spectra, wherein (B) depicts the effect of temperature, sample immersed in water, and wherein (C) depicts the dry film spectrum compared to that in water, where both were taken at 25° C., where the spectra within each panel are for the same experimental configuration, and where the same film was used for the measurements; and

FIG. 12 illustrates the synthesis of compound 4 (N-[(S-(2′chloropropionylethylester) N′N′ diethyldithiocarbamate))]-N-methyl-6-thioctic amide), in accordance with embodiments of the present invention.

### DETAILED DESCRIPTION OF THE INVENTION

In some embodiments, the present invention is directed to methods by which nanoparticle interactions can be controlled, compositions with which such interactions can be controlled, and devices which utilize the control of such interactions. Generally, such methods involve binding or grafting polymer to electromagnetically-functional cores to form a core/shell composite nanoparticle, assembling a plurality of such core/shell nanoparticles to form an assembly, and exposing the assembly to at least one environmental stimulus to which the polymer is responsive so as to modulate the interparticle interactions of the electromagnetically-functional cores. The present invention is also directed to the compositions resulting from such methods and to the methods and associated devices for controlling the interparticle interactions in such compositions.

In the following description, specific details are set forth such as specific quantities, sizes, etc. so as to provide a thorough understanding of embodiments of the present invention. However, it will be obvious to those skilled in the art that the present invention may be practiced without such specific details. In many cases, details concerning such considerations and the like have been omitted inasmuch as such details are not necessary to obtain a complete understanding of the present invention and are within the skills of persons of ordinary skill in the relevant art.

Referring to the drawings in general, it will be understood that the illustrations are for the purpose of describing a particular embodiment of the invention and are not intended to limit the invention thereto.

“Electromagnetically-functional,” as defined herein, refers to a material that exhibits electromagnetic properties that are useful for applications involving such things as absorption or emission or reflection of wavelength in a part of the electromagnetic (EM) spectrum (typically visible, infrared for some specialized, e.g., military, applications), magnetic properties, electrical properties, and the like.

An “assembly,” as defined herein, is a structural arrangement of core/shell nanoparticles, wherein the shell is a polymer that is responsive to at least one environmental stimulus and which forms a matrix for the cores within the assembly, wherein the cores are subject to electromagnetic coupling, and wherein such coupling is initiated, cancelled, and/or modulated by the presence (or absence) of external stimuli. Such assemblies can be in the form of a continuous array, or they can reside in discrete regions within a larger array or film. The level of order within such assemblies and arrays can vary considerably, from unordered to highly ordered.

“Environmental stimulus,” as defined herein, is a stimulus to which the polymer shell or matrix is responsive upon exposure. Typically, such stimuli include, but are not limited to, heat, electromagnetic radiation, moisture, chemical stimuli (e.g., toxins), pH, electrical stimuli, and combinations thereof. Typically, the polymer response is one of expansion/contraction, so as to provide for modulation of interparticle spacing and coupling of the electromagnetically-functional cores. Depending upon the composition of the core/shell nanoparticles and on the type of environmental stimulus used, subjection of an assembly of core/shell nanoparticles to an environmental stimulus can result in an increase or decrease in electromagnetic coupling between the core/shell nanoparticles within the assembly.

Generally, the electromagnetically-functional cores can comprise any material and shape that provides for electromagnetic functionality, as described above. Such materials include, but are not limited to, metals, alloys, semiconductors, and combinations thereof. In some embodiments of the invention, suitable nanoparticle cores comprise gold, silver, copper, platinum, silicon, cadmium sulfide, cadmium selenide, and the like. In other embodiments suitable nanoparticle cores comprise AgBr, AgI, Cd₃As₂, Cd₃P₂, CdTe, GaAs, HgI₂, InAs, In₂S₃, In₂Se₃, PbS, PbSe, ZnO, ZnS, and ZnTe. In still other embodiments, suitable nanoparticle cores comprise sub-core/sub-shell particles (e.g., nanoshells) with a sub-shell selected from the group consisting of gold, silver, copper, silicon, cadmium sulfide, cadmium selenide and the like, and a sub-core comprising a substantially inert material such as silica or like material. In still other embodiments, suitable nanoparticle cores comprise sub-core/sub-shell particles with a sub-shell comprising a high band gap material, such as zinc sulfide, silica, and like materials, for passivation, and a sub-core comprising a semiconducting material such as cadmium sulfide, cadmium selenide and the like, or a metal such as gold, silver, copper, platinum, and the like. The average size of the nanoparticle cores is typically in a range of between about 1 and about 150 nanometers (nm), or in a range of between about 1 and about 100 nm, or in a range of between about 1 and about 50 nm, or in a range of between about 1 and about 30 nm, or in a range of between about 1 and about 20 nm, or in a range of between about 1 and about 10 nm, as measured, for example, by dynamic light scattering (DLS) or transmission electron microscopy (TEM). Typically, the majority of nanoparticle cores are spherical or approximately spherical, although there is no particular limitation of the nanoparticle shape, and in some embodiments suitable nanoparticles may be ellipsoidal, or irregularly shaped, or rod-shaped (e.g., nanorods).

Generally, any polymer that is suitably responsive to at least one environmental stimulus can be used as the shell in the core/shell nanoparticles and associated compositions. In some particular embodiments, examples of suitable polymers include, but are not limited to, those comprised of structural units derived from at least one alkylacrylamide, wherein the alkylacrylamide can be selected from the group consisting of N-isopropylacrylamide (NIPA), N-(meth)acryloylpiperidine, N-ethylacrylamide, N-propylacrylamide, N,N-diethylacrylamide, N-cyclopropylacrylamide, N-acryloylpyrrolidine, N,N-ethylmethylacrylamide and N-ethylacrylamide; at least one alkyl(meth)acrylamide, wherein the alkyl(meth)acrylamide can be selected from the group consisting of N-isopropyl(meth)acrylamide, N-(meth)acryloylpiperidine, N-ethyl(meth)acrylamide, N-propyl(meth)acrylamide, N,N-diethyl(meth)acrylamide, N-cyclopropyl(meth)acrylamide, N-(meth)acryloylpyrrolidine, N,N-ethylmethyl(meth)acrylamide and N-ethyl(meth)acrylamide; at least one N-vinylamide such as, but not limited to, N-vinylisobutyramide; and combinations thereof. Some suitable polymers comprise structural units derived from N-isopropylacrylamide. Some suitable polymers are comprised of structural units derived from at least one vinyl alcohol. Some suitable polymers are comprised of acrylates, such as acrylic acid. Some suitable polymers comprise methacrylates, such as, but not limited to, 2-hydroxyethylmethacrylate. Some suitable polymers comprise elastin-like polypeptides. Additionally or alternatively, acrylonitrile may be used. Both homopolymers and copolymers may be employed. Suitable copolymers include, but are not limited to, those comprising structural units derived from at least one of any member from the list above, such as: N-alkyl(meth)acrylamide or at least one N-vinylamide in combination with a minor amount of at least one other monomer selected from the group consisting of N-acryloylglycine, acrylic acid, acrylamide, 2-aminoethylmethacrylate hydrochloride, and vinylimidazole. Homopolymers and copolymers that may be employed as stimulus-responsive hydrogels optionally also comprise structural units derived from at least one crosslinking monomer to prevent the hydrogel from dissolving in water. Suitable crosslinking monomers include, but are not limited to, N,N′-methylenebisacrylamide, methylenebismethacrylamide and ethyleneglycoldimethacrylate, with N,N′-methylenebisacrylamide being preferred. When present, crosslinker is typically used in an amount between about 4 and 15% of monomer weight, more preferably about 5% of monomer weight.

Generally, the polymeric shell is disposed on the cores with either or both of non-specific binding of polymers to the cores at multiple points along the polymer chains and end-grafting of polymer chains to the cores via linker moieties at the ends of the polymer chains. In some embodiments, the binding involves an interaction selected from the group consisting of covalent bonding, ionic bonding, chemisorption, physisorption, and combinations thereof. “Non-specific binding,” as used herein, refers only to van der Waals-based interactions, such as physisorption. All other types of binding in the aforementioned list are examples of specific binding mechanisms which drive binding through the presence of specific functional groups and are responsible for end-grafting. Another mechanism for end-grafting-like binding is when a block copolymer is used, and one block has a stronger non-specific interaction with the surface than the other block. In this case, the block copolymer can appear end-grafted, with one block on the surface of the particle like a train, and the other block emanating from it outwardly.

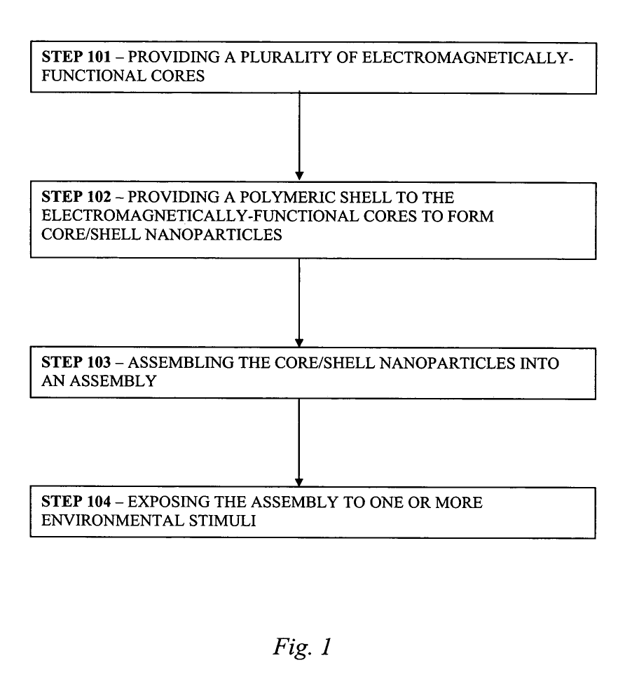

Referring to FIG. 1, in some embodiments the present invention is directed to a method comprising the steps of: (Step 101) providing a plurality of electromagnetically-functional nanoparticle cores; (Step 102) providing a polymeric shell to each of the electromagnetically-functional cores to form a plurality of core/shell nanoparticles, wherein the polymeric shell is responsive to at least one environmental stimulus, and wherein the polymeric shell is bound to the electromagnetically-functional core in a manner selected from the group consisting of binding at sites along the length of the polymeric chain, end-grafting involving binding at the ends of the polymer chains, and combinations thereof; (Step 103) assembling the plurality of core/shell nanoparticles into an assembly in which the electromagnetically-functional cores are subject to being electromagnetically coupled to each other; and (Step 104) exposing the assembly to at least one environmental stimulus so as to modulate the extent to which the electromagnetically-functional cores are electromagnetically coupled to each other.

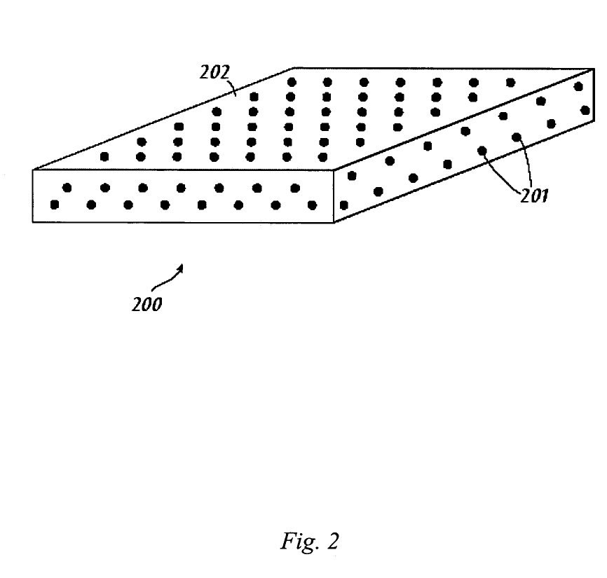

Referring to FIG. 2, such an above-described continuous assembly 200, in accordance with some embodiments of the present invention, typically comprises a plurality of electromagnetically-functional nanoparticle cores 201 in a polymer matrix 202. The polymeric matrix controls an interparticle separation between the electromagnetically-functional cores throughout the assembly, wherein the minimum separation between core surfaces achievable by the polymer shell upon stimulation is of up to about 50 nm. In some embodiments, the minimum separation is up to about 20 nm, in some embodiments it is up to about 10 nm, and in some embodiments it is up to about 5 nm. The shape of such an assembly is not particularly limited, but responsiveness to environmental stimuli is generally enhanced with greater surface area. An exemplary shape or form is a film. As mentioned above, such an assembly need not be continuous and can, for example, be a part of a larger, organized array. Moreover, the electromagnetically-functional nanoparticle cores need not be distributed throughout the assembly or array in an ordered fashion.

In some embodiments, the present invention is directed to a composition comprising: (a) at least one electromagnetically-functional core having a diameter in a range from about 1 nm to about 100 nm; and (b) a polymeric shell disposed on an outer surface of the electromagnetically-functional core and substantially covering the electromagnetically-functional core, wherein the polymeric shell is responsive to at least one environmental stimulus, and wherein the polymeric shell is bound to the electromagnetically-functional core in a manner selected from the group consisting of binding at sites along the length of the polymeric chain, end-grafting involving binding at the ends of the polymer chains, and combinations thereof. Typically, the polymeric shell has a shell thickness in the range of about 0.5 nm to about 20 nm for the unswollen state.

In some embodiments, the above-described composition is made by: (a) providing at least one electromagnetically-functional core; and (b) binding or grafting polymer to the electromagnetically-functional core via non-specific binding through the chain and/or grafting to the end of the polymer through specific binding, wherein such non-specific binding includes, but is not limited to, van der Waals attractive forces; such specific binding includes, but is not limited to, covalent bonding, ionic bonding, hydrogen bonding, and combinations thereof; and wherein such grafting includes the use of linker moieties. Suitable linker moieties for grafting include, but are not limited to, thiol linkages, disulfide linkages, phosphine linkages, phosphine oxide linkages, carboxylic acid linkages, amine linkages, phosphate linkages, sulfate linkages, isocyanate linkages, siloxane linkages, and the like.

In some embodiments, the present invention is directed to a film comprising: (a) a stabilized polymeric matrix, wherein the stabilized polymeric matrix is responsive to at least one environmental stimulus; and (b) an assembly of electromagnetically-functional cores disposed in the matrix, each of the electro-magnetically-functional cores having a diameter in a range from about 1 nm to about 100 nm, wherein the electromagnetically-functional cores are typically monodisperse (but not necessarily so), substantially unagglomerated, and electromagnetically coupled to each other, and wherein the stabilized polymeric matrix controls an interparticle separation between the electromagnetically-functional cores throughout the film. Note that electromagnetically-functional cores need not be monodisperse, and in some embodiments it is advantageous that they not be (vide infra). “Stabilization,” as defined herein and as it relates to a stabilized polymeric matrix, is any physical or chemical treatment that renders the polymeric matrix insoluble in solvents such as water. For example, in some embodiments the outermost end of the polymer shell has a UV-sensitive group that is activated upon UV irradiation. This activation leads to a polymerization across the shells of adjacent composite core/shell nanoparticles, thereby setting or stabilizing the assembly in place and rendering it stabilized.

In some embodiments, the above-described film is formed by: (a) providing a plurality of electromagnetically-functional cores; (b) providing a polymeric shell to each of the electromagnetically-functional cores to form a plurality of core/shell nanoparticles; (c) assembling the plurality of core/shell nanoparticles into an assembly in which the electromagnetically-functional cores are subject to electromagnetic coupling with each other; and (d) stabilizing the assembly to form the film. In some embodiments, the step of assembling involves a casting technique. Suitable casting techniques include, but are not limited to, drop casting, spin casting, etc.

In some embodiments, the present invention is directed to a sensor, the sensor comprising a film, wherein the film comprises: (a) a stabilized polymeric matrix, wherein the stabilized polymeric matrix is responsive to at least one environmental stimulus; and (b) an assembly of electromagnetically-functional cores disposed in the matrix, each of the electromagnetically-functional cores having a diameter in a range from about 1 nm to about 100 nm, wherein the electromagnetically-functional cores are typically monodisperse (but not necessarily so), substantially unagglomerated, and electromagnetically coupled to each other, wherein the stabilized polymeric matrix controls an interparticle separation between the electromagnetically-functional cores throughout the film, wherein the sensor inspects an absorbed, emitted, or reflected intensity and/or wavelength of radiation by the film both before and after exposure to the stimulus, and wherein a change in the wavelength absorbed, emitted, or reflected by the film indicates the presence of the stimulus.

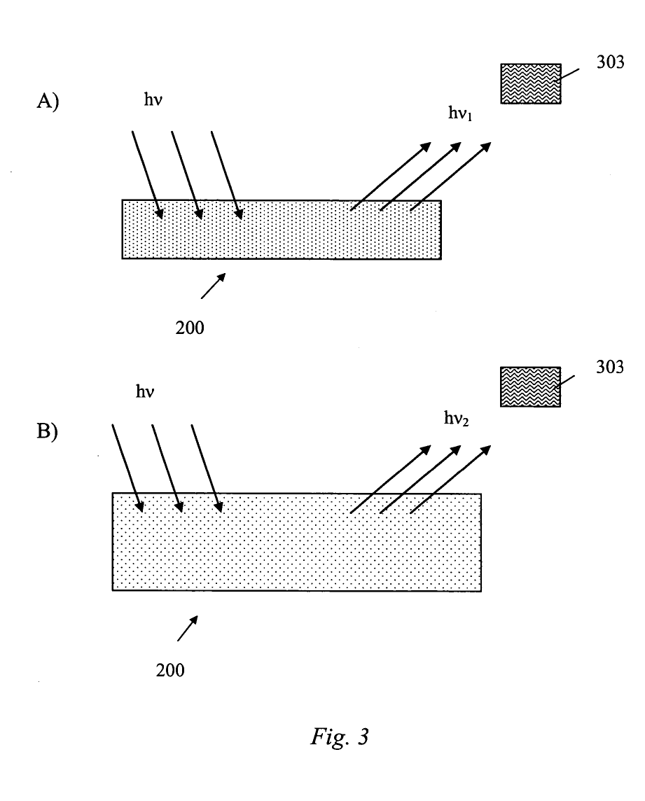

FIGS. 3A and 3B depict how such an above-described representative sensor operates. FIG. 3A depicts an interaction of assembly (film) 200 with EM radiation in the absence of an environmental stimulus to which the polymer matrix is responsive. Interacted radiation emanates from the film as hv₁. FIG. 3B depicts the interaction of such an assembly 200 with EM radiation in the presence of such a stimulus, such that the interacted radiation hv₂ differs in wavelength/frequency from that of hv₁, as determinable with detector 303.

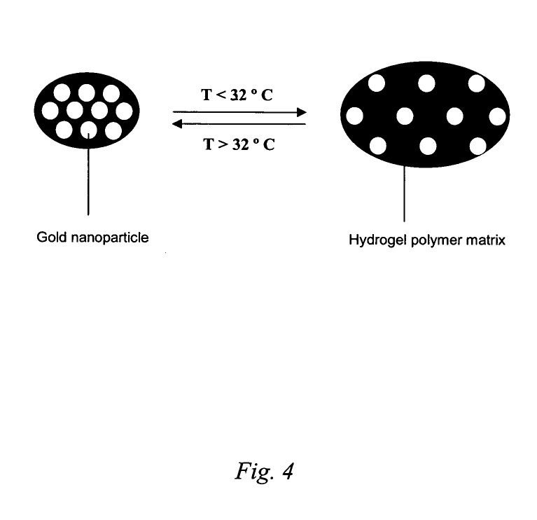

In some particular embodiments, the present invention is directed to methods that afford dynamic control of interparticle coupling within a single material, demonstrating reversible, on-demand switching of optoelectronic properties for the case of a gold nanoparticle assembly. An exemplary dynamic medium for controlling interparticle coupling in such embodiments is poly(N-isopropylacrylamide) (pNIPA), a temperature-sensitive polymer known to undergo large reversible size changes of up to a 1000-fold in volume (Tanaka, Phys. Rev. Lett., 1978, 40:820). This embodiment is illustrated schematically in FIG. 4. This reversibility can be extended to other systems that fall within the scope of embodiments of the present invention.

In contrast to previous approaches using stimulus-responsive pNIPA for controlling interparticle coupling (Sheeney-Haj-Ichia et al., Adv. Funct. Mater., 2002, 12:27; Pardo-Yissar et al., Adv. Mater., 2001, 13:1320), the above-mentioned particular embodiments rely on generating composite core/shell particles with well-defined size through the control of pNIPA shell thickness. There are numerous benefits to such an approach.

A first benefit of the above-described particular embodiments is the ability to create highly dense particle arrays that exhibit optical features of classical coupling between particles, i.e., changes in not only color, but also metallic luster (Farbman et al., J. Phys. Chem., 1992, 96:8469; Farbman et al., J. Chem. Phys., 1992, 96:6477; Weller, Angew. Chem. Int. Ed. Engl., 1996, 35:1079; Murray et al., Science, 1995, 270:1335; Coe et al., Nature, 2002, 420:800; Collier et al., Science, 1997, 26:1978; Storhoff et al., J. Am. Chem. Soc., 2000, 122:4640; and Redl, Nature, 2003, 423:968). Although such effects have long been observed through studies of metal liquid-like films and Langmuir monolayers, they have not been previously demonstrated to occur reversibly in a freestanding coating suitable for application on a substrate (Farbman et al., J. Phys. Chem., 1992, 96:8469; Farbman et al., J. Chem. Phys., 1992, 96:6477; Collier et al., Science, 1997, 26:1978). In contrast to previous approaches reported to yield only 0.006 wt. % gold, the particular embodiments described herein afford films having high gold density, as low as 1-10 wt. % for the swollen state and as high as 50 wt. % for the shrunken state, based on a calculated estimate for the average film thickness. Furthermore, films obtained via such particular embodiments are stable and resistant to delamination for at least up to 50 swelling/shrinking cycles tested thus far, whereas in the previous art, delamination was reported to occur at the maximum achievable gold loading of only 0.006 wt. % (Sheeney-Haj-Ichia et al., Adv. Funct. Mater., 2002, 12:27; Pardo-Yissar et al., Adv. Mater., 2001, 13:1320).

A second benefit of using composite core/shell particles in accordance with the above-described particular embodiments of the present invention is the ability to control shell functionality through the incorporation of chemical substituents into the polymer. For example, Applicants have initiated studies aimed at understanding the relationship between polymer binding mode and bulk film properties such as optical contrast, as well as reversibility and kinetics of optical switching. Accordingly, results from initial studies at the single particle level are included herein. Additionally, the ability to control particle size enables the formation of ordered particle assemblies, thereby providing controlled interparticle coupling. There is no particular limitation on the particle size distribution of the nanoparticles.

In some embodiments, both the core nanoparticles and the composite core/shell nanoparticles may possess a broad particle size distribution, while in other embodiments they may possess a narrow average particle size distribution. In one particular embodiment, such nanoparticles possess a narrow average particle size distribution with less than 5% size dispersion, meaning that less than 5% of the population lies on either side of the mean particle size value. In another particular embodiment, such nanoparticles may possess a bimodal size distribution achieved, for example, by combination of two different sizes of nanoparticles synthesized separately. In illustrative embodiments wherein at least two different nanoparticle populations each possessing a different average particle size are blended together, then the ratios of said populations may be in a range of about 90:10 to about 10:90. The stimulus-responsive polymer shell enables reversible control of the magnitude of the interparticle spacing, serving as an on/off switch between two distinct optoelectronic states of the assembly. In these ways, material functionality can be controlled through several levels of structural hierarchy, with the added capability of remotely controlling interparticle coupling within a single material.

In some embodiments, assemblies of the core/shell nanoparticles of the present invention find application beyond that of film-based sensors. Such applications include, but are not limited to, novel aesthetics (e.g., aesthetic coatings), functional paints, etc. in consumer electronics and medical equipment applications.

The following examples are included to demonstrate particular embodiments of the present invention. It should be appreciated by those of skill in the art that the methods disclosed in the examples that follow merely represent exemplary embodiments of the present invention. However, those of skill in the art should, in light of the present disclosure, appreciate that many changes can be made in the specific embodiments described and still obtain a like or similar result without departing from the spirit and scope of the present invention.

### EXAMPLE 1

This Example serves to illustrate both non-specific binding and grafting of polymer to core nanoparticles, and serves to illustrate the efficacy of disulfide linkages vs. thiol linkages in favoring one architecture over the other in binding polymer to gold nanoparticle cores, in accordance with some embodiments of the present invention.

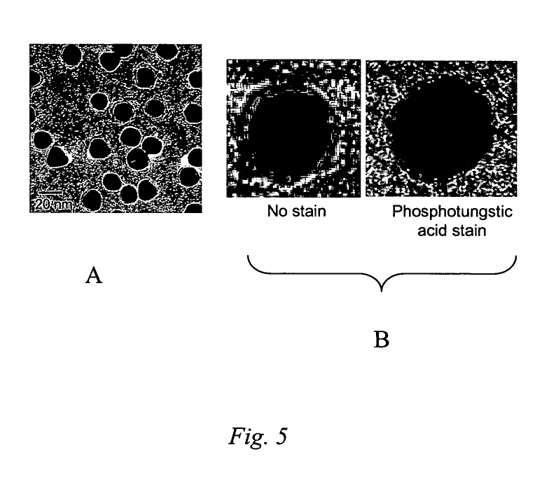

The graft-to approach for particle functionalization is a more generally applicable technique, allowing each constituent of the composite core/shell particle to be prepared separately by well-established techniques, potentially allowing control of both particle size and polymer molecular weight (Zhu et al., J. Am. Chem. Soc., 2004, 126:2656; and Mangeney et al., J. Am. Chem. Soc., 2002, 124:5811). The pNIPA polymers used in this Example were prepared using the reversible addition fragmentation chain transfer (RAFT) method with the goal of providing polymer shells of defined thickness (Schilli et al., Macromolecules, 2002, 35:6819). Gold core particles were prepared by the citrate method because this method affords electrostatically-stabilized particles of narrow size distribution in water, and it enables surface grafting of pNIPA through facile surface exchange of weakly bound surface citrate ions (for preparation of the citrate-stabilized gold, see D. A. Handley in Colloidal Gold: Principles, Methods, and Applications, Vol. 1 (Ed: M. A. Hayat), Academic Press, Inc., New York, 1989, Ch. 1). Subsequent particle functionalization occurs under mild conditions by simply mixing the two constituents in solution and allowing preferential polymer adsorption onto the gold particles to take place (note that all glassware for the gold colloid preparation was cleaned with aquaregia and thoroughly rinsed with deionized water). In a typical preparation, approximately 30-50 mg of polymer were added to 30 mL of aqueous citrate-Au (freshly prepared) and left to stir overnight in the dark. The resulting solution typically appeared unchanged in color (red) or slightly brownish-red, depending on the pNIPA derivative used. The product was isolated by centrifugation (˜4° C.; 30 000 g) and the resulting pellet was washed with de-ionized water 3 times, isolating the product by further centrifugation between washes. The collected washes, typically faint red in color, were discarded. The final product was isolated as an intensely red colored solution and stored as an aqueous solution in the refrigerator (>0° C.). Particles prepared in this way were well-dispersed core/shell structures (composite nanoparticles), with little to no aggregation present based on transmission electron microscopy. Example images are shown in FIGS. 5A and 5B, wherein FIG. 5A is a TEM image of the gold/pNIPA core/shell particles showing that they are well dispersed, and wherein FIG. 5B is a TEM image of the gold/pNIPA core/shell particles where the core/shell structure is more clearly apparent.

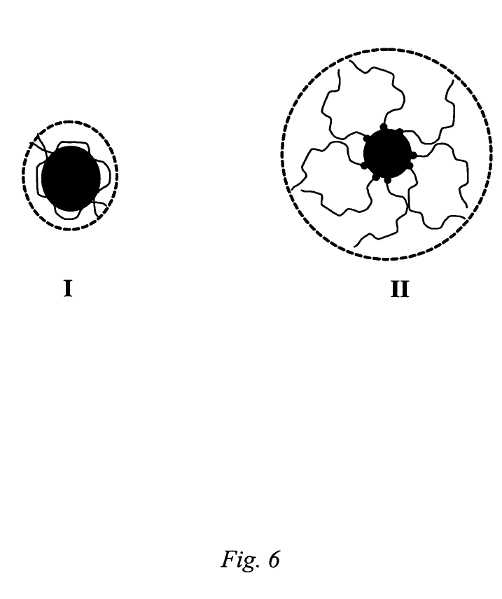

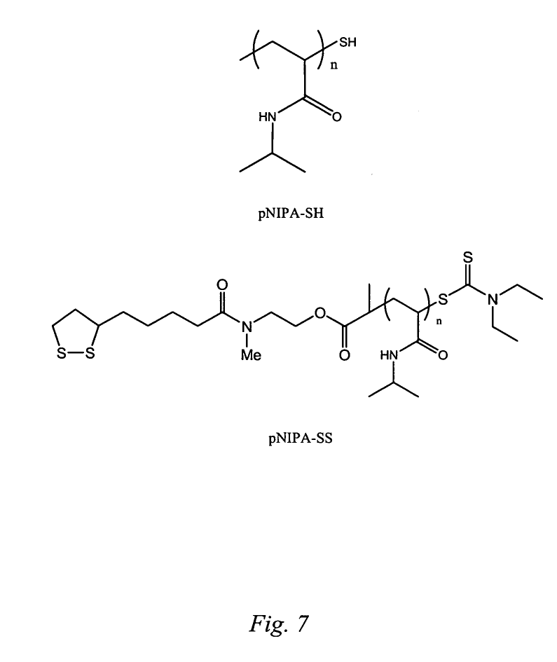

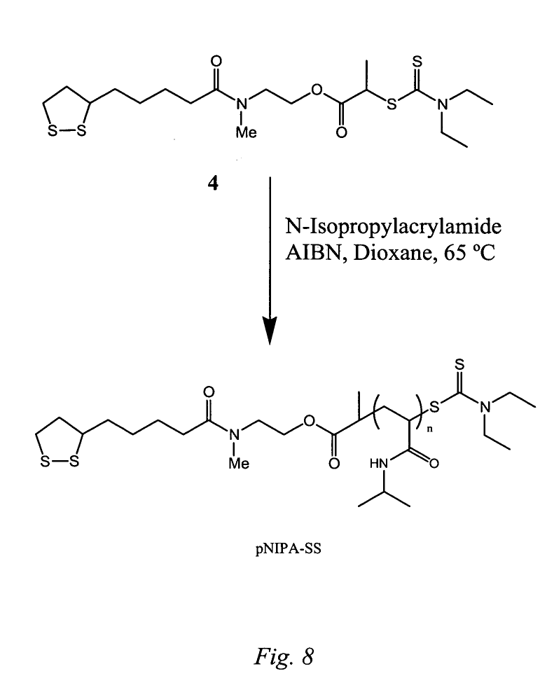

The architecture of the pNIPA graft to the particle surface is expected to affect the maximum size-change of the polymer shell and associated kinetics. Therefore, Applicants have initiated investigation of variously end-functionalized pNIPA samples, with the goal of generating two distinct surface-bound polymer architectures, I and II, corresponding to non-specifically bound polymer and end-grafted polymer, respectively, as shown in FIG. 6. The pNIPA obtained via standard AIBN-initiated free radical polymerization was used for generating architecture I. Referring to FIG. 7, two different sulfur-based chain-end functionalities, thiol for PNIPA-SH, and disulfide for pNIPA-SS were chosen to compare their efficacies in driving the end-grafted architecture, II (note that all pNIPA-SH polymers were obtained from Polymer Source, Inc., Dorval, Canada). FIG. 8 shows the reaction for generating pNIPA-SS used for generating architecture II. 15K PNIPA-SS was prepared according to Schilli et al., Macromolecules, 2002, 35:6819 (Mn 15K; PDI 1.5; GPC in THF using polystyrene standards). While both chain-end functionalities (moieties) are sulfur-based to facilitate surface-binding to gold, they differ critically in that pNIPA-SS features an additional hydrophobic spacer between the disulfide anchor and the pNIPA chain that is expected to be more effective in driving end-binding of water-soluble pNIPA chains from aqueous solution (R. J. Hunter in Foundations of Colloid Science, Vol. 1, Oxford University Press, New York, 1995, Ch. 8). In solution stability studies (e.g., salting studies), both PNIPA- and pNIPA-SH-coated gold particles (pNIPA/gold and pNIPA-SH/gold) were found to behave similarly; therefore, Applicants focused further studies on the comparison between pNIPA-SH and pNIPA-SS derivatives. Described below are initial results on the effects of polymer structural features (chain-end functionality and molecular weight) on the thermally-induced size change of the composite core/shell particles. Based on these studies, the pNIPA-SS sample was chosen for film preparation and corresponding UV-vis characterization.

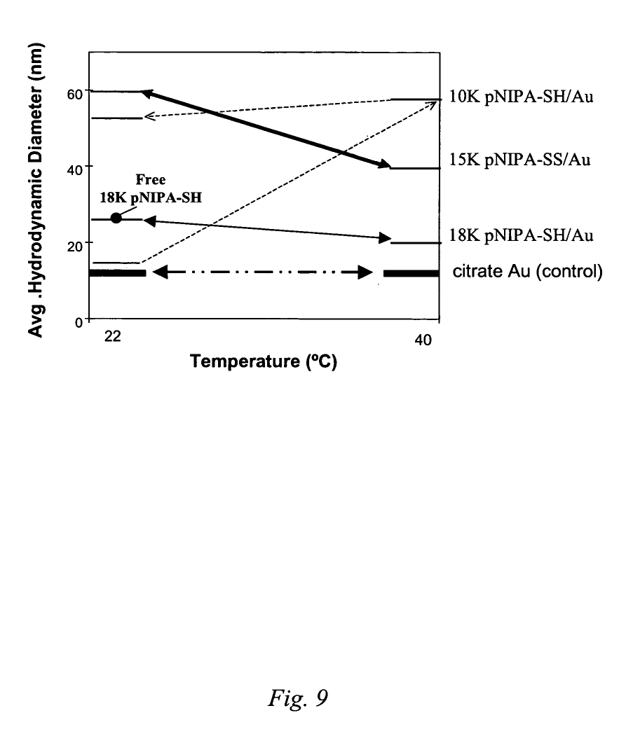

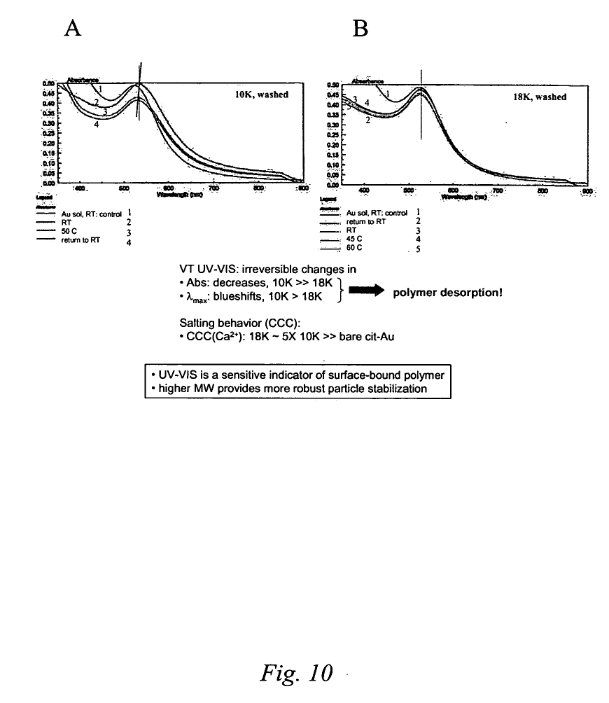

Dynamic light scattering (DLS) was used to infer the particle shell architecture and also to observe the reversible size change of the variously coated pNIPA/gold nanoparticles upon heating above 32° C., the critical solution temperature of pNIPA. Dynamic light scattering was performed using a Brookhaven Instruments BI-200SM goniometer. A Melles Griot HeNe laser (633 nm) was used. The sample cell, a glass test tube, was contained in a constant temperature bath of vat fluid, decalin, index matched to the glass sample cell. The vat fluid was filtered through a 0.2 μm filter to remove dust. The temperature of the vat fluid was maintained by a recirculating bath fluid, which heated and cooled a plate beneath the vat fluid bath as necessary. The detector was an avalanche photodiode, with the output signal processed by a BI-9000AT digital correlator. Correlation functions were measured over delay times ranging from 0.1 μs to 1 sec and at a fixed angle of 90°. Correlation functions were collected for a duration that was 200 times longer than the largest reported delay time. Sample solutions were prepared for each measurement by diluting with 18 MΩ MilliQ water, followed by filtration using a 0.1 or 0.2 μm PTFE Whatman filter. For variable temperature studies, incubation at the desired temperature for ˜30 minutes was found to be amply sufficient for reaching equilibrium. All measurements were corrected for viscosity. For the series of particles examined, shown in FIG. 9 is a plot of the observed changes in average hydrodynamic diameter of the composite particle upon thermal cycling between 22° C. and 40° C. As expected, the control citrate gold particles are insensitive to this mild temperature change. For pNIPA-SH/gold, the observed behavior depends critically on the molecular weight of pNIPA-SH used: The 10K pNIPA-SH-gold sample can be seen to irreversibly increase in size upon thermal cycling, whereas the 18K pNIPA-gold sample shows the expected reversible size shrinkage. This opposing trend is interpreted as a difference in particle stability due to pNIPA molecular weight, and is further supported by variable temperature UV-vis and salting experiments shown in FIGS. 10A and 10B.

Referring again to FIG. 9, a comparison between 18K pNIPA-SH/gold and 15K pNIPA-SS/gold reveals a striking difference. While these samples both exhibit reversible size changes, they differ significantly in initial particle size (i.e., at room temperature). The 15K pNIPA-SS/gold is more than twice as big as the 18K pNIPA-SH/gold, despite their gold cores being identical (as confirmed by TEM and DLS). Furthermore, DLS analysis of free 18K pNIPA-SH polymer shows that its average coil size is comparable to that of the corresponding 18K pNIPA-SH/gold composite particle, strongly suggesting that the polymer is not end-grafted in the latter. Using the measured room temperature (22° C.) coil size of the free 18K pNIPA-SH as an estimate for the shell thickness in an end-grafted architecture, II, the corresponding composite particle size is expected to be just over 60 nm, nearly an exact match to the experimentally observed 15K pNIPA-SS/gold particle size. These observations, together with the similarities in solution stability of the pNIPA-SH/gold and pNIPA/gold, suggest that the architecture of the 18K pNIPA-SH/gold resembles that of I (non-specific binding), whereas the 15K pNIPA-SS/gold is more like that of II (end-on binding). Therefore, it appears that a thiol end-group alone may not be sufficient for driving the end-bound graft-to architecture.

Because of the larger fractional size change observed for the 15K pNIPA-SS/gold particles (˜33% vs ˜20% for 18K pNIPA-SH), this sample was deemed more suitable for preparing an assembled particle film to ensure maximum contrast between the two switchable states of the film. Purified particles were concentrated to approximately 1 mL from 10 mL by centrifugation and subsequent removal of the slightly pink tinted supernatant. The resulting deep red concentrate was then deposited onto a quartz slide, covered with a petri dish and left under ambient conditions, in the dark, until a dry (drop-cast) film remained (up to ˜48 hours). The drop-cast film prepared in this way was deep purple, and mirror-like in appearance. This film was subsequently exposed to a Xe 4.2-inch spiral lamp (Xenon Corporation) at 5.0 J/cm² (1/2 J per 3 seconds, for ca. 30 seconds). A labile photo/thermal dithiocarbamate end-group incorporated into the 15K pNIPA-SS polymer as a result of the RAFT polymerization mechanism potentially enables surface-confined chain-end coupling between neighboring particles, thereby stabilizing the assemblage in place, onto the quartz substrate. It should be noted that confining cross-polymerization to the particles' surfaces is important for maintaining the large size change of the end-grafted pNIPA shell. Increasing crosslink density within hydrogels has been shown to substantially reduce the magnitude of this response.

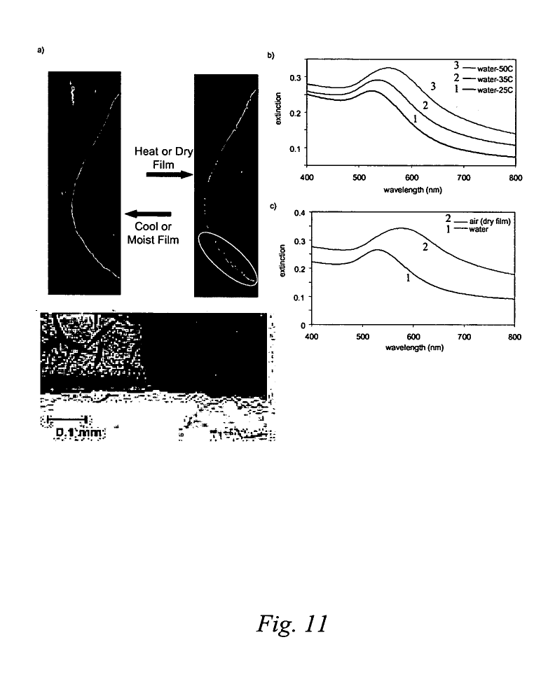

The resulting stabilized composite film is both moisture and temperature sensitive, consistent with the properties of the hydrogel. FIGS. 11A-C show the effect of temperature on the appearance of the film. In its dry state, the film is purple and mirror-like, whereas in the presence of water at room temperature, the film becomes red and transparent, losing its mirror-like appearance. If the film is heated above ˜32° C. while immersed in water, it reverts to its dry, purple appearance. This reversible switching behavior was observed beyond 50 cycles, showing no film delamination. In contrast, films of pNIPA/Au (architecture I), did not exhibit reversible behavior beyond 1-2 cycles, after which these films remained irreversibly purple in color. The 15K pNIPA-SS/Au-based film was investigated further by variable temperature UV-vis studies (UV-vis spectra were obtained on a double beam Cary 500 instrument equipped with a Peltier attachment. A sample of film on a quartz slide was inserted in a glass solution cell. For variable temperature studies in aqueous solution, the sample was allowed to equilibrate for about 30 minutes before obtaining a spectrum). Shown in FIGS. 11B and 11C are the corresponding extinction spectra for the temperature (B) and moisture sensitivity (C) taken for the same film. When the film is dry or heated, the gold plasmon absorbance maximum is red shifted, by up to 50 nm when a wet film is heated, and by up to 70 nm when a moist film is dried. Overall, extinction is increased, but most efficiently at longer wavelengths. Most significant, however, is the absence of secondary features, such as a shoulder in the red tail that is typical of uncontrolled aggregation (Mangeney et al., J. Am. Chem. Soc., 2002, 124:5811; and Lazarides et al., J. Phys. Chem. B, 2000, 104:460). Additionally, little peak broadening occurs in the variable temperature study, and the peaks are nearly congruous with respect to one another through their red tails.

The above-described observations suggest that the interparticle separation is well controlled throughout these assemblies and that the thermally-induced change in interparticle (core) separation occurs coherently throughout the film (Collier et al., Science, 1997, 26:1978; Lazarides et al., J. Phys. Chem. B, 2000, 104:460). When the films are taken to complete dryness (FIG. 11C), however, significant peak broadening does occur.

Thus, this Example demonstrates a method for making a new class of nanostructured composites featuring switchable optical properties through remote control of interparticle interactions. Unaggregated core/shell particles with stimulus responsive polymer shells of controlled thickness are the key building blocks for generating controlled interparticle separations that govern the optical properties of this novel composite material. The grafting architecture of the polymer shell is expected to have a strong effect on the magnitude and kinetics of the stimulus-driven size change of the composite particle, and therefore a key material design feature that should be the focus of further studies. Initial results indicate that a thiol end-group is not sufficient to drive end-grafting of pNIPA chains to the gold particle surface, yet the pNIPA-SS, having a disulfide moiety effectively linked to the pNIPA chains via a small spacer (approx. equivalent to 12 repeating methylenes), appears to be effective in obtaining the end-grafted architecture. Furthermore, based on UV-vis spectroscopy of thin films, it has been observed that the change in interparticle separation occurs coherently throughout the film-unlike previous studies in which uncontrolled aggregation is believed to occur. The approach is general in that stimulus-responsive shells of controlled thickness could be applied to other nanoparticle systems for varying electronic or magnetic properties of their bulk assemblies.

### EXAMPLE 2

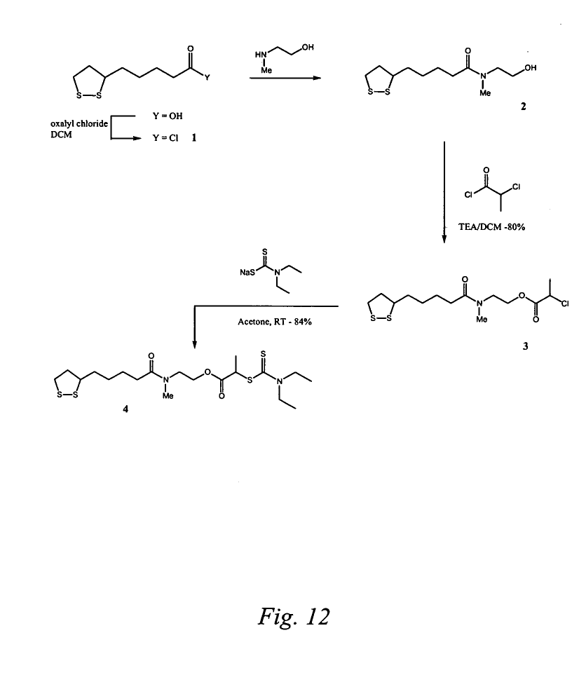

This Example serves to illustrate the synthesis of compound 4 used in the preceeding Example and as depicted in FIG. 12.

### A) Materials

Reagents were purchased from Aldrich and used as received unless otherwise indicated. Anhydrous solvents were obtained from Aldrich. pNIPA-SH samples were purchased from Polymer Source, Inc.

### B) Characterization

All nuclear magnetic resonance (NMR) spectra were obtained on a Bruker Avance 400 equipped with a 5 mm H/C dual probe. All spectra were obtained using standard parameters supplied with Bruker's XWINN software. These included a 30° flip angle, 1 second pulse delay, 10 kHz spectral width for proton and 30 kHz spectral width for ¹³C. Polystyrene standards in the range of 10 kD to 300 kD were used to establish a calibration. The molecular weight determination of pNIPA was carried out using ambient temperature gel-permeation chromatography (GPC) using a HP model 1050 LC system in-line to a HP model 1050 UV detector and a Varex model ELS II A evaporative light scattering detector (ELSD). The chromatography was achieved using an isocratic elution with a mobile phase of 100% tetrahyrofuran (THF) (LC grade). Approximately 1.5 mg of each sample was placed into a sampling vial along with 2 mL of THF. Each sample was then filtered through a 0.45 μm syringe filter. An injection volume of 50 μL was run through a Polymer Labs gel mixed column system. Instrumental parameters were as follows: flow rate: 1.0 mL min⁻¹; columns: 2-PL-gel Mixed-B® 300×25 mm GPC columns (10 μm pore size/10⁴ Å to 500 Å); solvent system: 100% THF (LC grade); UV Detection at 280 nm. ELSD parameters: nebulizer pressure was 55 psi nitrogen/temperature was 108° C. Mass spectra for small molecules were acquired using a JEOL model HX-110, high resolution magnetic mass spectrometer. The mass spectrometer was operated at 1000 resolution with a scan rate of 1 scan/sec. The sample was introduced into the mass spectrometer using a solids probe that was heated linearly to about 200° C. Electron ionization (EI) was used to produce ions. UV irradiation was done with a Xenon 4.2-inch Spiral Lamp (Xenon Corporation). The sample was exposed at 50 J/cm² (0.5 J per 3 seconds, for ca. 30 seconds). Matrix-assisted laser-desorption ionization time-of-flight (MALDI-TOF) mass spectra were acquired on a Applied Biosystems Voyager DE-STR mass spectrometer equipped with a standard nitrogen laser. The analyzer was operated in either linear or reflectron mode. A typical sample preparation of pNIPA for MALDI-TOF is as follows. About 10 mg of pNIPA was dissolved in 1 mL of THF (LC grade). A 1.5 μL aliquot of polymer solution was transferred to a conical vial, and 25 μL of matrix solution (10 mg/mL solution of 2-(4-hydroxyphenylazo) benzoic acid (Aldrich 14,803-2, used without additional purification) (HABA) in THF) was then added to the vial. The vial was then mixed on a vortex mixer for 30 seconds. Approximately 0.1 μL of the solution was used to spot the standard stainless steel flat MALDI plate. The solution was dispensed very slowly to minimize spot spreading on the plate.

### C) Thioctic Acid Chloride (1)

Thioctic acid chloride (1) was prepared using oxalyl chloride in dichloromethane (DCM) (see Sabapathy, R. C.; Bhattacharyya, S.; Leavy, M. C.; Cleland, W. E.; Hussey, C. L. Langmuir 1998, 14, 124-136. Thioctic acid N-methyl-N-2-hydroxyethylamide (2) was prepared according to a literature procedure (see Laschewsky, A.; Rekaï, E. D.; Wischerhoff, E. Macromol. Chem. Phys. 2001, 202, 276-286).

### D) N-(2′chloropropionylethylester)-N-methyl-6-thioctic Amide (3)

A 3.48 g (13.2 mmol) sample of 2 was dissolved in 130 mL of DCM and 2.0 mL of triethylamine (TEA) was added. The solution was cooled down to ca. 0° C. and 1.84 g (14.6 mmol) of 2-chloropropionyl chloride was added dropwise via syringe. The reaction stirred under nitrogen overnight. The crude material was purified by flash chromatography using a mixture of hexanes:ethyl acetate as the eluent. The final product was isolated as an oil (4.47 g, 10.6 mmol, 80%). ¹H NMR (400 MHz, CDCl₃): δ 3.2-2.9 (m, 3H), 2.5-2.2 (m, 3H), 2.0-1.4 (m, 5H), 1.3-0.9 (m, 3H), 0.65 (m, 1H), 0.5-0.1 (m, 9H). MS (EIMS) m/z calcd for (C₁₄H₂₄ClNO₃S₂) 353.09, found 353.

### E) N-[(S-(2′chloropropionylethylester) N′N′diethyldithiocarbamate))]-N-methyl-6-thioctic Amide (4)

A 4.47 g (12.7 mmol) sample of 3 was dissolved in 130 mL of dry acetone and 3.00 g (13.3 mmol) of sodium diethyldithiocarbamate trihydrate. The reaction stirred overnight under nitrogen at room temperature. Acetone was removed by rotary evaporation and the crude reaction mixture was purified by flash chromatography using a hexanes:ethyl acetate mixture as the eluant (4.97 g, 10.7 mmol, 84%). ¹H NMR (400 MHz, CDCl₃): δ 4.75 (m, 1H), 4.4-4.2 (m, 2H) 4.0 (q, 2H), 3.8-3.5 (m, 5H), 3.2-2.9 (m, 5H), 2.5-2.2 (m, 3H), 1.92 (m, 1H), 1.8-1.4 (m, 9H), 1.4 (dd, 6H). ¹³C NMR (100 MHz, CDCl₃): δ 193.41, 193.11, 172.91, 172.31, 63.91, 62.50, 56.46, 49.65, 49.07, 48.85, 48.09, 47.01, 40.23, 38.48, 36.91, 34.79, 33.70, 33.24, 32.79, 29.05, 24.66, 17.17, 12.57, 11.54. HRMS (EIMS) m/z calcd for (C₁₉H₃₄N₂O₃S₄) 466.1452, found 466.1487.

### F) Poly(N-isopropyl acrylamide) (pNIPA-SS)

A sample of 1 g of N-isopropyl acrylamide was charged into a Schlenk flask followed by 0.32 mL (1.95 μmol) of an azoisobutyronitrile (AIBN)/dioxane stock solution (6.1 mM) and 0.913 mL (0.020 mmol) of chain transfer agent (CTA)/dioxane stock solution (21.5 mM). The total volume was adjusted to 5 mL with additional Dioxane. Following three freeze-pump-thaw cycles, the reaction was sealed under argon and stirred for 24 hours at 70° C. The reaction mixture was cooled to room temperature, and precipitated twice in petroleum ether and air dried overnight (0.513 g, 51%). ¹H NMR (400 MHz, DMSO-d6): δ 7.5-7.0 (br, 1H), 3.85 (s, 1H), 2.2-0.8 (m, 9H). MS (GPC−THF) Mw=15K, PDI=1.6. MALDI-MS (SS-M₂₂-dit: SS-M₂₂, and M₂₂ were all observed). Poly(N-isopropyl acrylamide) (pNIPA-SH 6K). MS (GPC−THF) Mw=10K (PDI=2.0). Poly(N-isopropyl acrylamide) (pNIPA-SH 29K). MS (GPC−THF) Mw=18K (PDI=2.3).

In summary, the present invention provides methods by which nanoparticle interactions can be controlled, compositions with which such interactions can be controlled, and devices which utilize the control of such interactions. Generally, such methods involve binding or grafting polymer to electromagnetically-functional cores to form a core/shell composite nanoparticle, assembling a plurality of such core/shell nanoparticles to form an assembly, and exposing the assembly to at least one environmental stimulus to which the polymer is responsive so as to modulate the interparticle interactions of the electromagnetically-functional cores. The present invention also provides compositions resulting from such methods, and devices resulting from such compositions.

It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above- described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

What is claimed is:

1. A method comprising the steps of:
a) providing a plurality of electromagnetically-functional cores;
b) providing a polymeric shell to each of the electromagnetically-functional cores to form a plurality of core/shell nanoparticles, wherein the polymeric shell is responsive to at least one environmental stimulus, and wherein the polymeric shell is bound to the electromagnetically functional core in a manner selected from the group consisting of non-specific binding at sites along the length of the polymeric chain, end-grafting involving non-specific binding at the ends of the polymer chains, and combinations thereof;
c) assembling the plurality of core/shell nanoparticles into an assembly in which the electromagnetically-functional cores are subject to being electromagnetically coupled to each other; and
d) exposing the assembly to at least one environmental stimulus so as to modulate the extent to which the electromagnetically-functional cores are electromagnetically coupled to each other.

2. The method of claim 1, wherein the electromagnetically-functional cores comprise metals, alloys, semiconductors, and combinations thereof.

3. The method of claim 1, wherein the electromagnetically-functional cores comprise diameters from about 1 nm to about 100 nm.

4. The method of claim 1, wherein the polymeric shell comprises pNIPA.

5. The method of claim 1, wherein the core/shell nanoparticle has a size controlled by the thickness of the polymer shell.

6. The method of claim 1, wherein the polymer shell is bound to the electromagnetically-functional core with binding that comprises end-grafting, wherein such end-grafting comprises linkages selected from the group consisting of thiol linkages, disulfide linkages, phosphine linkages, phosphine oxide linkages, carboxylic acid linkages, amine linkages, phosphate linkages, sulfate linkages, isocyanate linkages, siloxane linkages, and combinations thereof.

7. The method of claim 1, wherein the step of assembling comprises a casting of the core/shell nanoparticles into a film.

8. The method of claim 1, wherein the step of exposing comprises exposure to an environmental stimulus selected from the group consisting of heat, EM radiation, moisture, chemical stimuli, pH, electrical stimuli, and combinations thereof.

9. A composition comprising:
a) at least one electromagnetically-functional core having a diameter in a range from about 1 nm to about 100 nm; and
b) a polymeric shell disposed on an outer surface of the electromagnetically-functional core and substantially covering the electromagnetically-functional core, wherein the polymeric shell is responsive to at least one environmental stimulus, and wherein the polymeric shell is bound to the electromagnetically-functional core in a manner selected from the group consisting of non-specific binding at sites along the length of the polymeric chain, end-grafting involving binding at the ends of the polymer chains, and combinations thereof.

10. The composition of claim 9, wherein the electromagnetically-functional core comprises a form selected from the group consisting of a nanoparticle, a nanoshell, and combinations thereof.

11. The composition of claim 9, wherein the electromagnetically-functional core comprises Au.

12. The composition of claim 9, wherein the polymer shell comprises pNIPA.

13. The composition of claim 9, wherein the binding involves an interaction selected from the group consisting of covalent bonding, ionic bonding, chemisorption, physisorption, and combinations thereof.

14. The composition of claim 9, wherein the composition comprises at least two electromagnetically-functional cores, and wherein the polymer is operable for controlling interparticle distance between the at least two cores in response to environmental stimuli.

15. The composition of claim 9, wherein the composition is made by a method comprising the steps of:
a) providing an electromagnetically-functional core; and
b) binding the polymer to the electromagnetically-functional core via binding selected from the group consisting of covalent bonding, ionic bonding, hydrogen bonding, van der Waals attractive forces, and combinations thereof.

16. The composition of claim 15, wherein the polymer is bound to the electromagnetically-functional core in a manner involving end-grafting, and wherein the end-grafting comprises chemical linkers selected from the group consisting of thiol linkers, disulfide linkers, phosphine linkers, phosphine oxide linkers, carboxylic acid linkers, amine linkers, phosphate linkers, sulfate linkers, isocyanate linkers, siloxane linkers, and combinations thereof.

17. The composition of claim 9, wherein the composition is operable for use in applications selected from the group consisting of consumer electronics, medical equipment, and combinations thereof.

18. The composition of claim 17, wherein such use involves aesthetic coatings, functional paints, and combinations thereof.

19. A film comprising:
a) a stabilized polymeric matrix, wherein the stabilized polymeric matrix is responsive to at least one environmental stimulus; and
b) an assembly of electromagnetically-functional cores disposed in the matrix, each of the electromagnetically-functional cores having a diameter in a range from about 1 nm to about 100 nm, wherein the electromagnetically-functional cores are substantially unagglomerated and subject to being electromagnetically coupled to each other, and wherein the stabilized polymeric matrix controls an interparticle separation between the electromagnetically-functional cores throughout the film.

20. The film of claim 19, wherein the film is formed by:
a) providing a plurality of electromagnetically-functional cores;
b) providing a polymeric shell to each of the electromagnetically-functional cores to form a plurality of core/shell nanoparticles;
c) assembling the plurality of core/shell nanoparticles into an assembly in which the electromagnetically-functional cores are subject to being electromagnetically coupled to each other; and
d) stabilizing the assembly to form the film.

21. The film of claim 20, wherein the step of assembling involves a casting of a dispersion of the core/shell nanoparticles.

22. A sensor, the sensor comprising a film, wherein the film comprises:
a ) a stabilized polymeric matrix, wherein the stabilized polymeric matrix is responsive to at least one environmental stimulus; and
b) an assembly of electromagnetically-functional cores disposed in the matrix, each of the electromagnetically-functional cores having a diameter in a range from about 1 nm to about 100 nm, wherein the electromagnetically-functional cores are substantially unagglomerated and subject to being electromagnetically coupled to each other, and wherein the stabilized polymeric matrix controls an interparticle separation between the electromagnetically-functional cores throughout the film, wherein the sensor monitors changes in radiation, after having interacted with the film, both before and after exposure of the film to the stimulus; wherein such changes in radiation are selected from the group consisting of (i) changes in wavelength of the radiation, (ii) changes in intensity of the radiation, and (iii) combinations thereof; and wherein such changes in the radiation are indicative of a stimulus being present.

23. The sensor of claim 22, wherein the sensor is responsive to environmental stimuli selected from the group consisting of heat, EM radiation, moisture, chemical stimuli, pH, electrical stimuli, and combinations thereof.

24. The sensor of claim 22, wherein the interaction of radiation with the film is observed by a manner selected from the group consisting of absorption, luminescence, reflection, and combinations thereof.
